(19) United States
(12) Patent Application Publication
(10) Pub. No.: US 2009/0221975 A1
(43) Pub. Date: Sep. 3, 2009

(54) ONE WAY VALVE

(75) Inventor: Aaron Leonard Rodd, Furleigh Waters (AU)

Correspondence Address: CANTOR COLBURN, LLP, 20 Church Street, 22nd Floor, Hartford, CT, 06103, US

(73) Assignee: MEDIGARD LIMITED, Queensland (AU)

(21) Appl. No.: 12/065,463

(22) PCT Filed: Aug. 21, 2006
(86) PCT No.: PCT/AU06/01203

§ 371 Date: Sep. 2, 2008

(30) Foreign Application Priority Data
Aug. 29, 2005 (AU) 2005904695
Oct. 18, 2005 (AU) 2005905748

(51) Int. Cl. A61M 25/14

(52) U.S. Cl. 604/256

(57) ABSTRACT

A medical valve can be attached to the front of a needle containing syringe or other needle containing medical device and can be operated between an open and closed position with the needle extending through the valve. A backflow prevention mechanism can be provided. The valve can be used with auto retracting needles.

### FIELD OF THE INVENTION

This invention is directed to a valve that can be used with or form part of a catheter or a cannula or with other devices that have fluid flow, and particularly devices that also include some form of puncture needle. The invention is also directed to this type of valve where the puncture needle can pass through the valve and the valve can still be operated between the open and closed position even with the needle extending through the valve. The invention is also directed to this type of valve that can prevent backflow. The invention is also directed to this type of valve that can be used together with a retractable needle to reduce needlestick injury. The valve may also be used without a needle.

### BACKGROUND ART

In the medical field, it is well known to use a cannula or a catheter to introduce fluid into, or withdraw fluid from, a patient. A cannula-type instrument is also widely used for body piercing (such as ear piercing).

A cannula/catheter typically comprises a small diameter hollow tubular sleeve which has a portion ultimately inserted into the vein of a patient. To do this, there is provided a puncture needle (typically steel). The puncture needle sits inside the tubular sleeve. The puncture needle projects out of the front of the sleeve and is pushed into a vein. The sleeve is then pushed forwardly and the puncture needle can be progressively retracted such that ultimately part of the sleeve is inside the vein. Typically, a nurse will then press against the vein to temporarily stop blood flow, will remove the needle entirely from the cannula/catheter, and will attach a small end cap. The pressure against the vein is then released to enable blood to flow into the hollow tubular sleeve. At that stage, a blood collection device (typically under vacuum) or a syringe or any other desired medical device can be attached to the other end of the cannula/catheter. This can allow blood to be removed from the person or medicines to be administrated to the person.

A catheter can also be used without the needle to drain fluids (urine, etc.) from the person.

It is known to provide a more sophisticated cannula/catheter which contains a valve. The valve is usually formed on the cannula/catheter and is typically a simple turn valve or a simple push valve. It is known to provide the cannula/catheter with a rear fitting or mounting (such as a luer lock or a tapered luer fitting) to enable a syringe or other device to be attached to the rear of the cannula/catheter. It is also known to provide side wings that can be used to enable the cannula/catheter to be taped to the person's skin to hold the device in place.

There are some disadvantages with these known devices. Some known devices, while having a valve, do not allow a puncture needle to pass through the valve. Thus, if a puncture needle is required, the valve must be positioned in such a manner that the operation of the puncture needle is not prevented.

In some other devices, the valve is designed in such a manner that, when the valve is open, there is an opening in the valve which is aligned with the cannula tube and a needle can be pushed through this opening and into the cannula tube. However, the problem with this arrangement is that the valve cannot be closed until after the needle has been retracted and this can cause potentially contaminated fluid to fill the cannula/valve or even spill out the back of the device.

Another disadvantage with these known devices is that fluids (for instance potentially contaminated bio fluids) pass through the valve when the valve is in the open position. While this, in itself, is not a disadvantage, the problem with these known devices is that when the valve is closed, an amount of fluid stays within the valve pathway or within the cannula/catheter tube. If the fluid is contaminated (e.g. HIV infected) this can create a source of infection for the medical practitioner. The fluid can also congeal or clot or otherwise block the tube.

Another disadvantage with many known devices is the relative complexity in the manufacture of the valve portion.

Another disadvantage with many known devices is that the operation of the valve between the open position and the closed position can be a two-handed affair, or requires movement that can be quite fiddly (it being appreciated that many of these devices can be quite small).

It is known to provide one-way valves that can be used in the medical field and which can also reduce or virtually eliminate backflow of potentially contaminated bio fluids. One way in which this can be achieved is to design the valve to have some form of backpressure when the valve moves from the open position to the closed position. For instance, it is known to provide the valve with some form of internal member that retracts when the valve moves to the closed position and, in the process, provide a positive pressure in the valve to prevent backflow. Indeed, such devices have been known for over 10 years. One difficulty with these devices is that the devices are designed in such a way that they cannot accommodate a puncture needle passing through the device. Thus, the device is not particularly suitable for use with an introducer (typically a steel puncture needle).

There are many medical situations where needlestick injury can occur. Needlestick injury can be a particular hazard if the needle is contaminated. Syringes, etc. all contain a needlestick hazard. It is considered that the introducer of a cannula/catheter comprises a particularly hazardous needlestick injury situation. For instance, the introducer typically comprises a steel puncture needle that needs to be inserted into the person's vein (for example). The cannula/catheter is then pushed forwardly and/or the needle is retracted from the person's vein. Ultimately, the needle is fully retracted from the cannula/catheter and because the needle has entered into the person's vein, the needle is always contaminated. It is considered that this comprises a particularly hazardous sharps risk.

For this reason, it is recently known to have an introducer with some form of shoot back needle. The introducer typically comprises a main body and a projecting needle and some form of retraction mechanism to trigger or retract the needle into the main body. A disadvantage with these known devices is that they are not suitable for use as an introducer of a catheter/cannula which contains an attached valve. Typically, the valve needs to be removed. Thus, although there is some improvement with having a retractable needle, as the valve cannot form part of the entire assembly and needs to be attached later on, there is still the risk of backflow of contaminated bio fluids through the cannula/catheter.

Therefore, there would be an advantage if it were possible to provide a valve that could enable a puncture needle to pass through the valve if required, and/or where the operation of the valve was relatively straightforward, and/or where there would be a reduction in retained bio fluids in the valve or the cannula/catheter when the valve is moved to the closed position.

There would also be an advantage if it were possible to provide an assembly which can comprise a cannula/catheter, a valve and a needle retraction device which can all work together to reduce the possibility of needlestick injury and reduce the possibility of bio fluids contaminating the nurse or medical practitioner inserting the cannula/catheter.

It will be clearly understood that, if a prior art publication is referred to herein, this reference does not constitute an admission that the publication forms part of the common general knowledge in the art in Australia or in any other country.

### OBJECT OF THE INVENTION

It is an object of the invention to provide a valve which may overcome at least some of the above-mentioned disadvantages or provide a useful or commercial choice.

In one form, the invention resides in a valve, and particularly a medical valve, the valve comprising a valve body having an inlet to allow a fluid to pass into the valve body, a valve member which is movable between a closed position where fluid is prevented from passing through the valve body, and an open position where fluid can pass through the valve body, the valve member including a seal, this seal being formed of resilient material which can be punctured by a needle to enable a needle to pass through this seal and through the inlet, and which is self-sealing upon removal of the needle.

In this manner, a puncture needle can pass through the valve and through the seal. When the puncture needle is retracted and removed from the valve, the seal is self-sealing and will close the hole formed by the puncture needle to enable the valve to remain in the closed position during insertion.

Suitably, the valve member comprises a forward portion which can contain the seal and a rear portion which is open. The rear portion may be configured to accept or to accommodate any type of medical device such as a syringe, tubing and the like and the rear portion may be configured with a luer lock or a tapered luer. Of course, any other type of suitable configuration may be used.

It is preferred that the valve member can move between the open position and the closed position in a sliding manner, and it is preferred that the valve member slides along an axis which is in line with or parallel to the longitudinal axis of the cannula/catheter. In this manner, operation of the valve can be a simple pull/push action which is in line with the longitudinal axis of the cannula/catheter, as opposed to many other valves which have a rotation action or a sliding action which is transverse to the longitudinal axis of the cannula/catheter.

An advantage with this type of “in-line” sliding operation of the valve is that the puncture needle (if used) can remain in the valve both in the open position and the closed position of the valve.

Connection makes the valve automatically open and removal males the valve automatically close, however the operation of connection and removal is not limited to this.

The valve member may be slideable within the valve body in such a manner that the valve member can slide but is restricted from rotation. An advantage of this is that a medical device can be twistlocked to the rear of the valve member without causing the valve member itself to rotate. This can be achieved by providing anti-rotating guide members. The anti-rotating guide members may comprise splines/rails/projections and the like which move along recesses or grooves and the like to allow a sliding movement but preventing rotational movement. Typically, the projections etc. may be provided on the valve member and the recesses etc. may be provided on the valve body. However, it is also envisaged that the projections etc. may be provided on the valve body, and the recesses etc. may be provided on the valve member, or any combination thereof.

Suitably, stop means and the like is provided to restrict complete withdrawal of the valve member from the valve body. The stop means may comprise a projection/shoulder and the like that can abut against a projection/shoulder or be retained by a recess and the like. Typically, the valve member has an outer wall containing the stop means and the valve body has an inner wall or an end portion and the like containing a stop means, the arrangement being that the valve member can be retracted to open the valve but this movement is limited to prevent the valve member from being pulled entirely out of the valve body.

Suitably, the valve member is retained in the closed position. This can be achieved by any suitable type of retention mechanism. In one form, the retention mechanism comprises a projection on the valve member which engages with or relative to part of the valve body to hold the valve member in the closed position.

To move the valve member to the open position, it will be necessary to overcome the retention mechanism. This can be achieved in various manners. In a preferred manner, the valve body can be deformed to release the retention mechanism thereby enabling the valve member to be moved (typically retracted) to the open position. Thus, the valve body may comprise a zone or area which can be deformed for instance by squeezing, to release the retention mechanism.

It is preferred that the valve is provided with some form of configuration or mechanism or means to reduce or preferably substantially eliminate any body fluids remaining in the valve when the valve is in the closed position.

In one form, this can be achieved by having a sliding valve member containing a forward sealing portion such that when the valve moves to the closed position, it can act somewhat alcin to a plunger within a syringe barrel and therefore expel any bio fluid in the valve such that when the valve member is in the closed position, the valve is substantially free from any bio fluid.

It is preferred that the valve forms part of a cannula/catheter, and therefore in a more particular form of the invention, there is provided a cannula/catheter comprising an elongate hollow tubular member adapted for insertion into a body cavity, the tubular member being attached to the valve.

In this more particular form of the invention, a puncture needle can pass through the valve and along the tubular member to facilitate insertion of the cannula/catheter into a body cavity or anywhere else.

An advantage of using the valve according to the present invention with a cannula/catheter is that one form of the invention enables the valve to have a positive pressure when the valve moves to the closed position to expel any bio fluid from the valve and also substantially from the tubular member which forms part of the cannula/catheter.

It should be appreciated that no particular limitation should be placed on the valve nearly by exemplifying a suitable use of the valve as with a cannula/catheter. That is, it is envisaged that the valve may also have other uses in other industries that require fluid flow to be regulated.

In another form, the invention can reside in an assembly comprising a valve which may be substantially as described above together with a cannula/catheter which is attached to the valve to form a single unit or a combined unit.

In another form, the invention can reside in an assembly comprising a valve and a cannula/catheter and also a puncture needle (also known as an introducer).

In another form, the invention can reside in an assembly comprising a valve, a cannula/catheter, a puncture needle and also comprising a needle retraction mechanism to enable the puncture needle to be safely retracted after use to reduce needlestick injury.

### BRIEF DESCRIPTION OF THE DRAWINGS

An embodiment of the invention will be described with reference to the following drawings in which:

FIG. 1 is a plan view of a cannula/catheter device containing a valve.

FIG. 2 illustrates a section view of the device of FIG. 1 with the valve in the closed position.

FIG. 3 illustrates a close-up view of the valve of FIG. 2 in the closed position.

FIG. 4 illustrates a close-up view of the valve in the open position.

FIG. 5 illustrates a transverse section view of the device of FIG. 1.

FIG. 6 illustrates a perspective view of the device of FIG. 1 showing attachment of a luer to the rear of the device.

FIG. 7 illustrates a section view of the device showing part of a rearwardly attached luer.

FIG. 8 illustrates a section view of the luer in greater detail.

FIG. 9 illustrates a part section of view of a valve according to a second embodiment of the invention attached to a cannula/catheter tube.

FIG. 10 illustrates a section view of the valve of FIG. 9 in the closed position.

FIG. 11 illustrates a section view of the valve of FIG. 10 in the open position.

FIG. 12 illustrates a part section view of the valve according to a third embodiment of the invention.

FIG. 13 illustrates a section view of the valve of FIG. 12 in the closed position.

FIG. 14 illustrates a section view of the valve of FIG. 13 in the open position.

FIGS. 15-17 illustrate an assembly comprising a valve, a cannula attached to the valve, a puncture needle (introducer) extending through the cannula, and a needle retraction mechanism attached to the rear of the valve.

FIGS. 18-20 illustrate various other uses of the valve other than simply for a catheter.

### BEST MODE

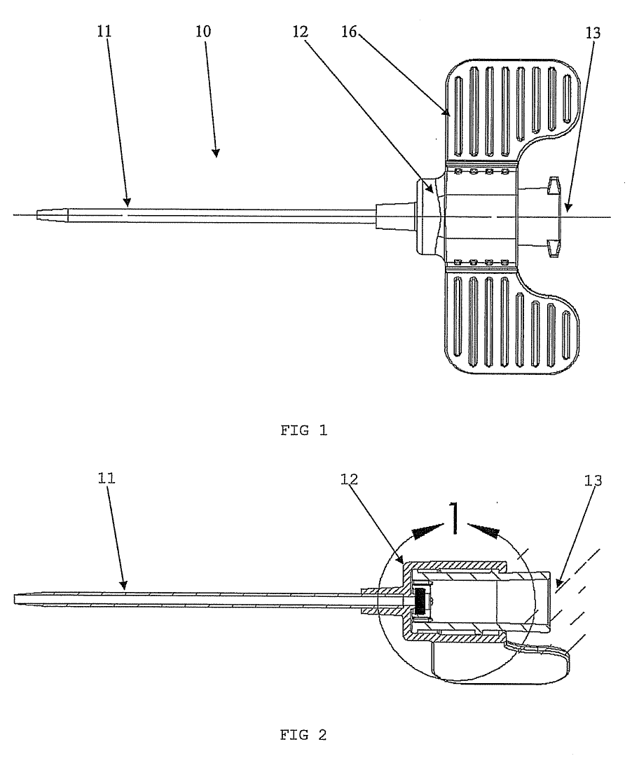

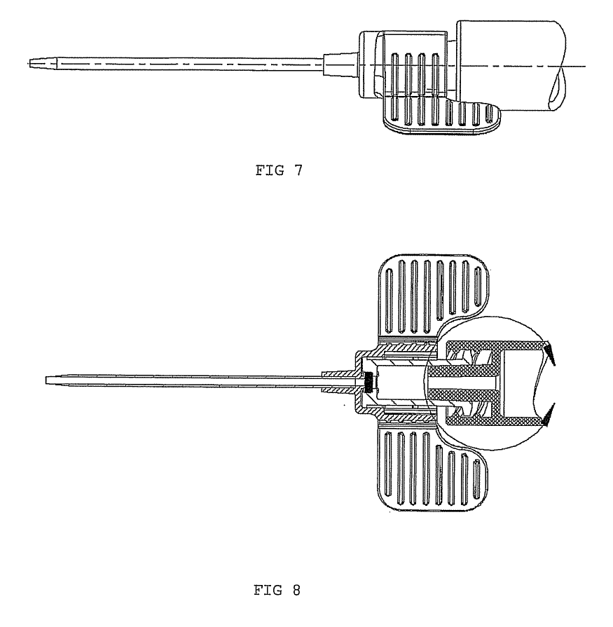

Referring to the illustrations, and initially to FIGS. 1 and 6, there is illustrated a cannula/catheter device 10 basically comprising a forwardly extending hollow tubular (typically plastic) member 11 (often called a needle although it is not a steel puncture needle), a valve 12 which is positioned at the rear of tubular member 11, the rear of valve 12 having an open end 13 and, in the particular embodiment, the open end 13 comprising a luer lock 14 to allow a luer 15 (for instance a syringe, etc.) to be fitted to lock 14. This itself is quite conventional. On each side of valve 12 is a relatively fiat wing 16, the function of these wings 16 being to provide a relatively large land area to enable plaster tape to hold the device securely to a person's skin. This is also quite conventional.

Referring to FIGS. 2-4, there is illustrated a section view particularly of valve 12. Also, these particular illustrations show the open end 13 containing a taper to enable a tapered luer to be press fitted to the open end. This itself is also quite conventional.

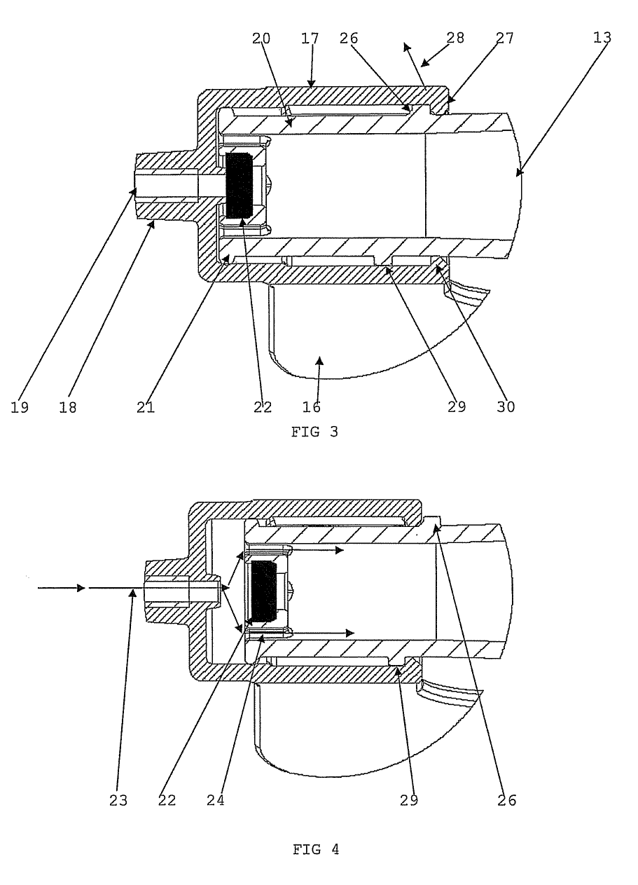

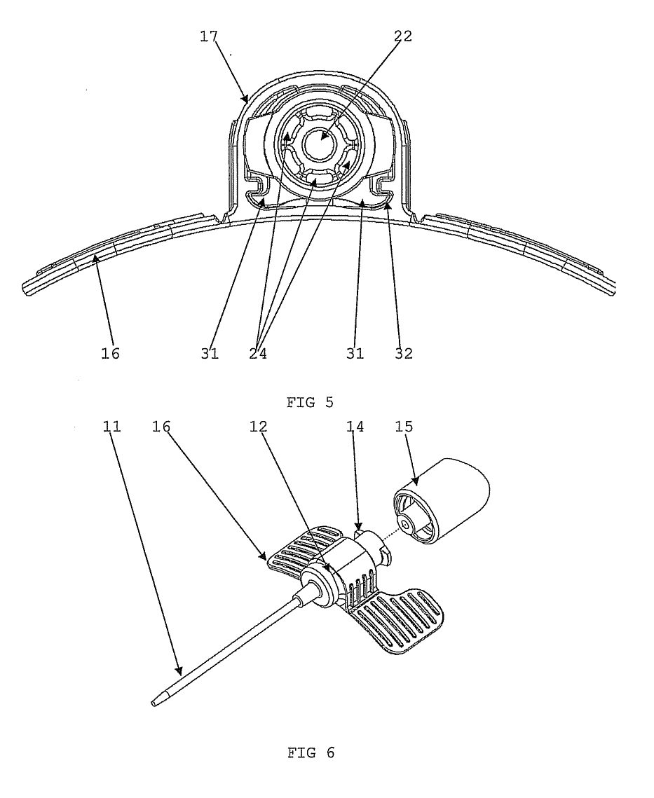

FIGS. 3-4 show the valve in the closed position (FIG. 3) and in the retracted open position (FIG. 4). Referring to these figures, the valve contains an outer body 17. A front portion of the outer body 17 contains a small diameter nose 18 with an internal passageway 19 into which the tubular member 11 can be fitted. Passageway 19 also forms the inlet of the valve. The forward part of outer body 17 is cylindrical and the rear part is somewhat oval to enable the sidewalls to be squeezed together, the reason for this being described in greater detail below.

Inside outer body 17 is a valve member 20. The valve member 20 is substantially cylindrical and hollow and contains a front end 21 which is in sealing or mainly sealing engagement with the inner wall of outer body 17. The rear end of the valve member 20 forms the open end 13 described previously.

The front end 21 also contains a central sealing member 22 which comprises a resilient self sealing rubber/plastic/silicone member. Pushing of valve member 20 into the forward position (FIG. 3) causes sealing member 22 to seal against inlet 17 to cause the valve to be in the closed position. Conversely, retraction of valve member 20 into the open position (FIG. 4) causes sealing member 22 to be spaced away from inlet 19 to allow fluid to flow through the inlet and along the fluid flow pathway illustrated as 23. To achieve the fluid flow pathway, a plurality of openings 24 (best illustrated in FIG. 5) is provided in the front face of valve member 20.

By having the sealing member 22 formed from self sealing material, a puncture needle (not illustrated) can be inserted through open end 13, pushed through sealing member 22 and along tubular member 11 to function as a puncture needle in the normal manner of a catheter or cannula. In this normal manner, once the puncture needle has punctured the body cavity (for instance a blood frame), the puncture needle can be entirely removed inter alia by pulling it back through sealing member 22, and as soon as the puncture needle has been removed from the sealing member, the sealing member will “self seal” to still provide a seal against passage of fluid through inlet 19 when the valve is in the closed position. These types of “self seal” sealing members are known in the art.

As mentioned previously, the valve member is operated in a sliding manner between the closed and open position and this movement is generally in line with the longitudinal axis of the entire device. This can provide ease of use.

Initially, the valve will be in the closed position illustrated in FIG. 3. In this position, the valve can not be inadvertently moved to the open position (for instance by backpressure). Therefore, the valve is retained in the closed position and needs to be forced into the retracted open position. This is achieved by providing a projection in the form of a shoulder 26 (see FIG. 3 and FIG. 4) extending outwardly from valve member 20 and which is retained by a turned in lip 27 on outer body 17. This prevents the valve from being inadvertently moved (retracted) to the open position illustrated in FIG. 4. To do so, it is necessary to deform the rear part of outer body 17. This can be done by squeezing the opposed ends of outer body together which will cause this part of the body to deform thereby slightly raising lip 27 in the direction of arrow 28 freeing shoulder 26 from engagement with the lip thereby allowing the valve to be retracted. Thus, in use, a medical practitioner will grip the rear part of outer body 17 and squeeze opposed walls together to free shoulder 26 from lip 27 to enable the valve member 20 to be retracted.

However, it is not desirable to easily allow the valve member to be completely pulled out of outer body 17. Therefore, another shoulder 29 is provided which is spaced inwardly relative to shoulder 26. The shoulder is also on the opposed side of valve member 20. The arrangement is that when the outer body 17 is deformed, this raises lip 27 in the region of shoulder 26, but does not deform the part of the outer body which is adjacent shoulder 29. Consequently, retraction of the valve member 20 can be done but only until shoulder 29 contacts its retaining lip 30 (see FIG. 4).

The valve member 20 is held in its sliding position and against rotation and lifting by a pair of rail members 31 (see FIG. 5). Rail members 31 comprise elongate members (rails) which are formed on a lower wall of valve member 20. The rails slide within recessed guides 32. The function of this arrangement is to prevent valve member 20 from rotating in outer body 17. For instance, if a twist lock medical device is attached to the open end 13, the twisting action can cause valve member 20 to rotate but this is prevented by the rail members 31. However, rail members 31 and recessed guides 32 are designed in such a manner that the valve member 20 is also prevented from “lifting” within outer body 17. To explain, when the outer body is squeezed to enable valve member 20 to be retracted, the squeezing action causes the top part of outer body 17 to deform (in the direction of arrow 28 in FIG. 3) which allows shoulder 26 to become free from lip 27. However, it is not desirable for the squeezing action to cause shoulder 29 to become free from lip 30. By having the particularly designed rails, the valve member 20 is held in such a manner that only shoulder 26 can become free to enable the valve to be retracted to the position illustrated in FIG. 4.

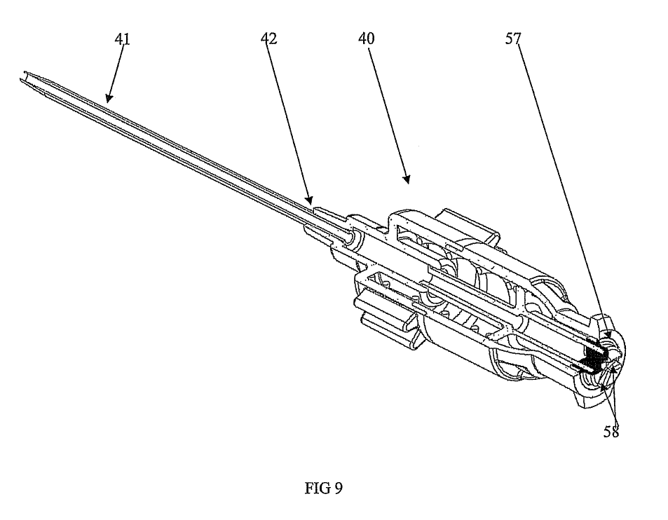

Referring now to the second embodiment of the invention illustrated in FIGS. 9-11, there is illustrated a valve and cannula combination. The combination comprises a valve 40 and a cannula 41 which is attached to the front of the valve. Cannula 41 will typically comprise a plastic tube having a degree of flexibility as is usual with cannulas/catheters. The length of the cannula can vary to suit. The rear end of the cannula is inserted into a front opening 42 of the valve and in any suitable manner to prevent the cannula from being pulled away from the valve.

The valve 40 comprises an outer body 43 which in the particular embodiment is made from two parts 44, 45 (see FIG. 10) which are fitted together and are required to enable the internal components to be assembled into the valve. The outer body has a front opening 42 and a rear opening 46. The outer body is substantially hollow. Inside the outer body is a slideable actuator 47 which has a particular design and which functions to open and close the valve. Actuator 47 can be made of plastic and comprises an elongate substantially hollow member having an open “downstream” front end 48 (which is closer to the valve outlet), and an “upstream” rear end (which is closer to the valve inlet) and which is closed by a rear seal 49. Actuator 47, by being substantially hollow, has a fluid passageway extending therethrough. Although the rear end of actuator 47 is closed by the rear seal 49, in front of rear seal 49 are a plurality of radially spaced ports 50 which extend through actuator 47 to communicate with the inside of outer body 43. Actuator 47 contains an intermediate disk-like portion 51 which sealingly engages against the inside wall of outer body 43 to provide an “intermediate seal”. Also, immediately adjacent the open front end 48 of actuator 47 is a front seal 52 which extends about the open front end 48.

As mentioned above, outer body 43 comprises part 44 (the forward part) and part 45 (the rear part). The forward part 44 contains a nose portion 53 and contains an internal cylindrical passageway 54 along which the front portion of the actuator 47 can slide.

A spring 55 is positioned inside outer body 43. Spring 55 in the particular embodiment comprising a helical spring. One end of the spring sits against the disk-like portion 51. The other end of the spring sits against an inside wall of forward part 44. The internal cylindrical passageway 54 provides a small internal “boss” 56 about which at least the part of spring 55 can be positioned to assist in holding the spring in place.

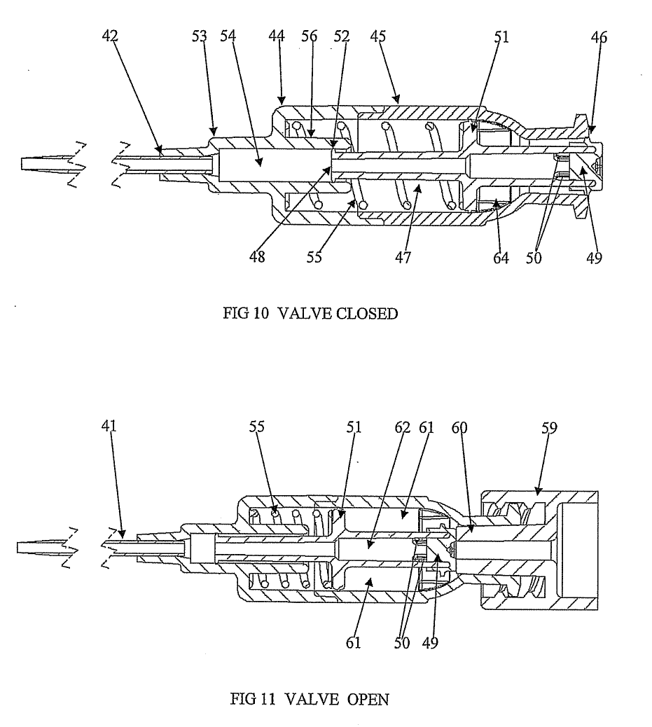

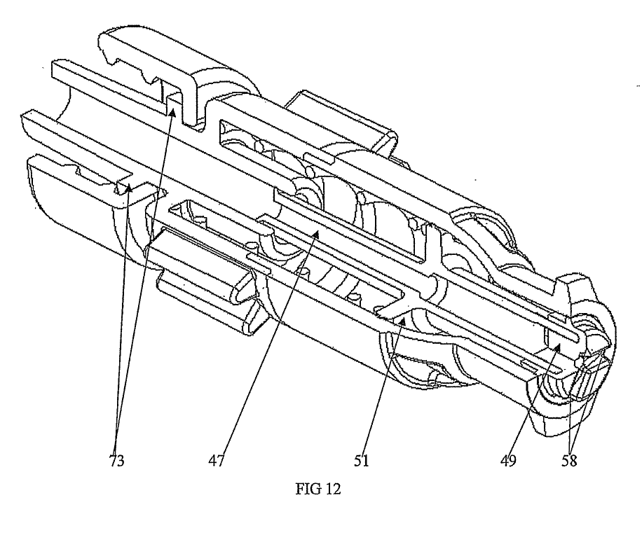

Spring 55 functions to push actuator 47 into the rear position (valve closed position) illustrated in FIG. 10. However, actuator 47 can be pushed into a forward position (valve open position) as illustrated in FIG. 11. Actuator 47 will be pushed (by spring 55) until the disk-like portion 51 strikes a support wall 64 inside the valve.

The rear seal 49 on actuator 47 is positioned to be at or adjacent the rear opening 46 when the valve is in the closed (rest) position. Rear seal 49 has a particular design and has a particular design of the outer face 57 which is best illustrated in FIG. 9 and FIG. 12. Referring to FIG. 9, the outer face 57 contains a number of slots or passageways 58. The reason for these passageways 58 is to enable fluid to flow past rear seal 49. This is best illustrated in FIG. 11. Referring to FIG. 11, this shows the valve in the open position where fluid can flow through the valve. The valve has been pushed to the open position (or more particularly, actuator 47 has been pushed forwardly in the valve) by attachment of a syringe 59 to the rear of valve 40. Of course, medical devices other than a syringe can be attached to the rear of valve 40. In this particular embodiment illustrated in FIG. 11, the syringe is attached in a conventional manner as illustrated and when this happens, the nose 60 of the syringe is pushed through the rear opening 46 which causes the actuator 47 to be pushed forwardly. The reason for the passageways of slots 58 on the front of rear seal 49 now becomes apparent as this enables fluid to flow from the syringe and along the passageways. In the absence of the passageways, rear seal 49 would seal against the front of the syringe to prevent fluid from flowing.

Referring both to FIG. 10 and FIG. 11, it can be seen that as the actuator is pushed forwardly, the rear seal 49 moves into the valve body and at the position illustrated in FIG. 11, the rear seal 49 no longer engages and seals against the inside wall of the valve body. At this position, fluid can flow through the syringe 59 along slots or passageways 58 and into the internal area 61 (see FIG. 11) of the valve body (internal area 61 can been seen as a fluid chamber).

Fluid will therefore fill up the internal area 61, but because actuator 47 contains the number of ports 50 extending through the wall of the actuator, fluid will also pass through the ports 50 and into the internal flow passageway 62 of actuator 47. The fluid can then flow along the flow passageway 62 and through the cannula 41.

When desired, the syringe 59 (or other medical device) can be removed from the rear end of valve 40. Because of spring 55, as soon as the medical device is removed, the spring will push actuator 47 back to the closed position illustrated in FIG. 10. Therefore, an advantage of this embodiment is that the valve is self-closing as soon as the syringe/medical device is removed. Put differently, there is no need to have a separate knob, etc. that needs to be rotated to close the valve.

The particular mechanism of the valve also prevents contaminated bio fluid from being sucked back along the cannula tube 41 and into the valve body. Any such bio fluid is highly undesirable and can also clot to prevent efficient working of the valve.

This backflow prevention is achieved as follows. As the syringe, etc. 59 is removed from the rear end of the valve, there will still be fluid in area 61 (see FIG. 11). As the actuator is pushed (by the spring) from the forward position illustrated in FIG. 11, the volume of this area will progressively reduce. Consequently, the fluid in area 61 will be “pumped into” flow pathway 62 through ports 50 and therefore back along cannula tube 41. This provides a backflow or backpressure of fluid and prevents contaminated bio fluids from being sucked into the valve which is not desirable.

Another advantage with the valve design is that a steel needle (for instance an introducer for a cannula/catheter) can be pushed through the valve and into the cannula 41. This means that the combination of the cannula, steel puncture needle and the valve can be packaged together in a sterile manner. Alternatively, the steel needle can be separately pushed through the valve. The reason why this can happen is by having the actuator flow pathway 62 substantially linear or straight. Also, the manufacture of seal 49 is from a “self sealing” composition through which the puncture needle can be pushed. Thus, it is possible to push the puncture needle through seal 49 and along flow passageway 62 and along the inside of cannula 41. This assembly can then be sterilised and packaged for use.

Another advantage of the valve design is that the needle can also be attached to a needle retraction mechanism. The needle retraction mechanism may comprise a mechanism that retracts the needle using vacuum, and such devices are described in our earlier patent applications. However, the needle retraction mechanism may also comprise a mechanism that can use a spring to shoot back or a spring to pull back the needle after use.

Thus, it is possible to provide a complete cannula/catheter and safety retraction needle assembly that can be sterilised and packaged. The assembly can comprise the cannula/catheter plus the valve as described above. The steel puncture needle can be fitted to a device that can be triggered to retract the steel puncture needle back into the body of the device after use to reduce or even completely eliminate the possibility of needlestick injury. The needle can be pushed through seal 49 and along the valve and along the cannula as described above.

This arrangement is illustrated generally in FIGS. 15-17. Referring to FIGS. 15-17, there is illustrated a cannula 41, a valve 40 which may be as described above, a puncture needle 66 which extends through valve 40 and along cannula 41 (the front tip of the puncture needle 66 being visible in FIG. 15) and a device 65 that can retract the needle using vacuum. The device 65 is best illustrated in FIG. 7 and comprises a vacuum chamber 66 and a forward portion 67 that can be attached to valve 40. Initially, puncture needle 66 is attached to a needle holder which is in the front of the device 65 and the needle 66 can then be pushed through valve 40, etc. to adopt the position illustrated in FIG. 15.

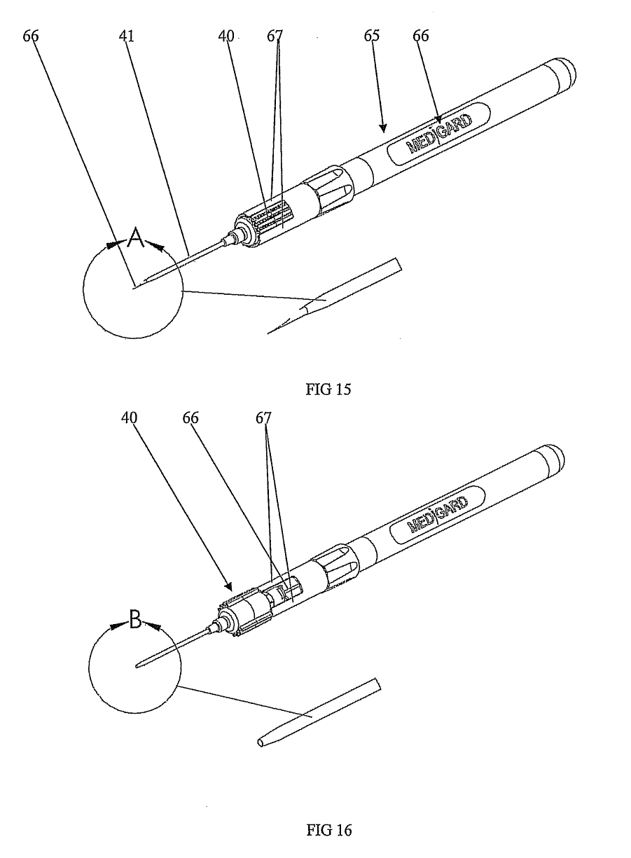

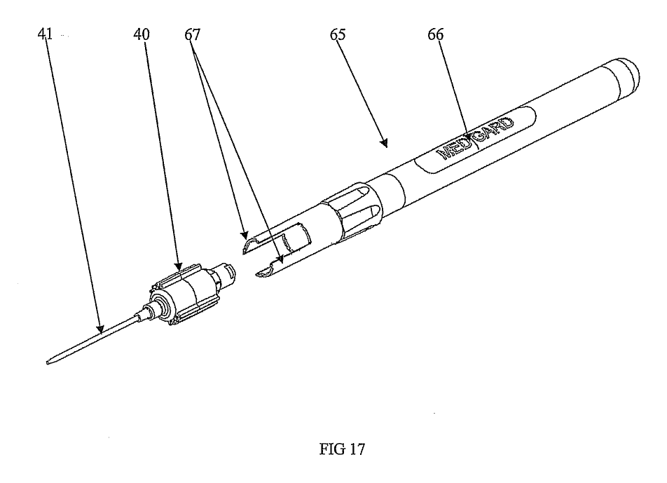

The needle can then be inserted into a person's vein and the cannula can be pushed off the needle and into the person's vein. It is common to also start pulling the needle out of the person's vein, and this can be done by starting to retract device 65 of the valve 40. This position is illustrated in FIG. 16 where it can be seen that the forward portion 67 has been pulled off valve 40. As soon as the needle has been sufficiently pulled out of the person's vein, device 65 can be triggered to retract the needle back within the vacuum chamber 66. This is the position illustrated in FIG. 17. Also, the device 65, while attaching to the rear of valve 40, does not push the actuator 47 into the forward (open) position. Thus, the valve 40 remains closed through the cannula introduction stage. Even as the needle is retracted into the vacuum chamber, the valve 40 remains closed.

Once the needle has been retracted and device 65 has been removed from the valve 40 (the position illustrated in FIG. 17), the valve can now be attached to a syringe, etc. in the manner illustrated in FIG. 11 to introduce fluid into the person's vein.

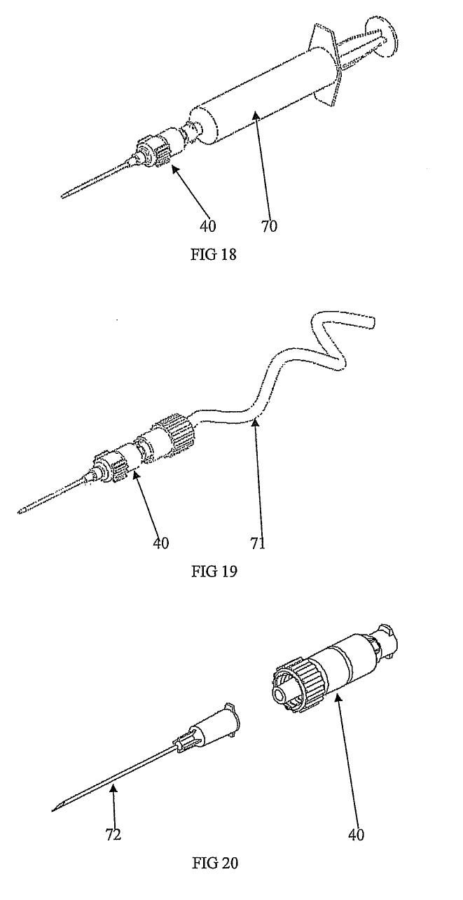

FIGS. 18-20 illustrate the various uses of valve 40. For instance, in FIG. 18, valve 40 can be attached to an ordinary syringe 70. In FIG. 19, valve 40 can be attached to a drip line 71. In FIG. 20, valve 40 is not attached to a cannula, but instead the front of the valve is designed to attached to a needle 72.

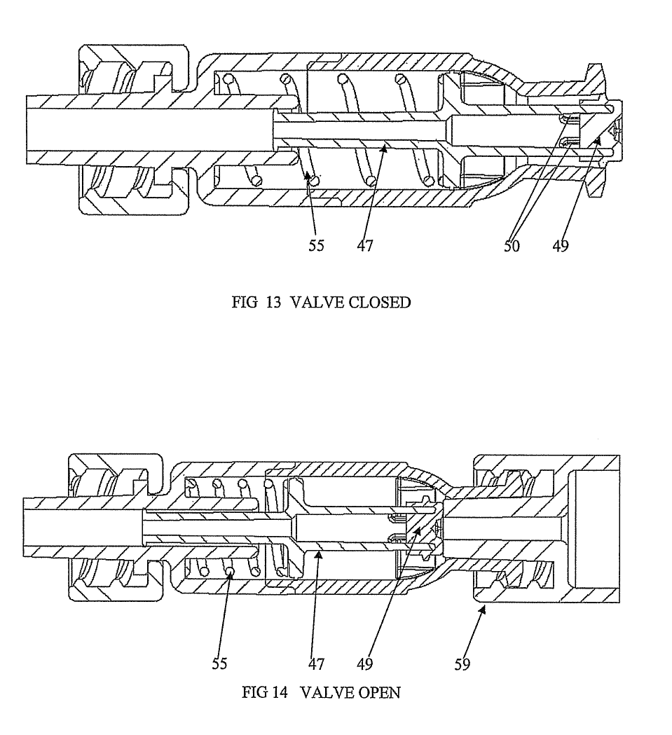

FIGS. 12-14 illustrate a variation to the valve design that make the valve suitable for attachment to a needle is illustrated in FIG. 20. Basically, the functioning of the valve is identical to that described with reference to FIGS. 9-11. However, the front of the valve contains a luer lock 73 to enable a luer to be fitted to the front of the valve. Of course, it should be appreciated that the front of the valve can also be tapered to form a press lock arrangement which is also known.

The valve according to the embodiment and a cannula/catheter containing the valve has many advantages. The valve has a simple linear open/closed action as opposed to a more complicated twisting or turning action. The particular action enables a positive pressure to be created upon closing the valve which will expel any residual fluids in the valve and the cannula/catheter tube. A puncture needle can be used with the valve and the puncture needle can be used when the valve is in the on position and in the off position. It also allows fluid to be injected or withdrawn from the patient via the cannula without the use of a needle (uses bare syringe or IV drip, etc.) It also allows the use of a needle if so desired, which can puncture the rubber seal/stopper without the need to open the valve. Because of the piercing ability while maintaining valve integrity, it can be used in conjunction with other devices. The valve can be produced with the cannula thereby eliminating the need to first insert cannula and then install the valve.

Throughout the specification and the claims (if present), unless the context requires otherwise, the term “comprise”, or variations such as “comprises” or “comprising”, will be understood to apply the inclusion of the stated integer or group of integers but not the exclusion of any other integer or group of integers.

Throughout the specification and claims (if present), unless the context requires otherwise, the term “substantially” or “about” will be understood to not be limited to the value for the range qualified by the terms.

It should be appreciated that various other changes and modifications can be made to any embodiment described without departing from the spirit and scope of the invention.

1. A medical valve comprising a valve body having an inlet to allow a fluid to pass into the valve body, an outlet to allow fluid to exit the valve, a valve member which is movable between a closed position where fluid is prevented from passing through the valve body, and an open position where fluid can pass through the valve body, the valve member including a seal which can be punctured by a needle to enable a needle to pass through this seal and through the inlet, and which is self-sealing upon removal of the needle, the valve member being operable between the open and closed position when the needle passes through the seal.

2. The valve of claim 1, wherein the valve member slides between the open position and the closed position.

3. The valve of claim 2, wherein insertion of a needle into the valve causes the valve to move to the open position and withdrawal of the needle causes the valve to move to the closed position.

4. The valve of claim 3, wherein a positive pressure is created in the valve body when the valve moves to the closed position to expel fluid in the valve body.

5. The valve of claim 4, wherein the valve member comprises a sliding actuator [47] which is elongate and contains a through passageway and which has an open front end [48] which is closer to the valve outlet and a rear end which is closer to the valve inlet, the rear end containing the seal [49] which can be punctured by the needle, the valve member further containing an intermediate seal [51], the area between the seal [49] and seal [51] defining a fluid chamber [61].

6. The valve of claim 5, wherein the fluid chamber defines a smaller volume when the valve is in the closed position and defines a larger volume when the valve is in the open position.

7. The valve of claim 6, including a spring to bias the sliding actuator to the closed position.

8. The valve of claim 7, wherein the sliding actuator is moved to the open position against the bias of the spring upon insertion of a medical device into the inlet of the valve body.

9. The valve of claim 6, wherein removal of the medical device from the inlet causes the sliding actuator to move under the bias of the spring to the closed position.

10. The valve of claim 9, wherein movement of the sliding actuator to the closed position causes reduction of the volume of the fluid chamber to provide positive pressure to fluid in the valve to expel fluid through the outlet and prevent backflow of fluid.

11. A cannula/catheter assembly comprising an elongate hollow tubular member adapted for insertion into a body cavity, the tubular member having a front opening and a rear opening, a valve comprising a valve body having an inlet to allow a fluid to pass into the valve body, an outlet to allow fluid to exit the valve, a valve member which is movable between a closed position where fluid is prevented from passing through the valve body, and an open position where fluid can pass through the valve body, the valve member including a seal which can be punctured by a needle to enable a needle to pass through this seal and through the inlet, and which is self-sealing upon removal of the needle, the valve member being operable between the open and closed position when the needle passes through the seal, the rear opening of the tubular member communicating with the outlet of the valve.

12. A cannula/catheter assembly comprising an elongate tubular member adapted for insertion into a body cavity, the tubular member having a front opening and a rear opening, a valve comprising a valve body having an inlet to allow a fluid to pass into the valve body, an outlet to allow fluid to exit the valve, a valve member which is movable between a closed position where fluid is prevented from passing through the valve body, and an open position where fluid can pass through the valve body, the valve member including a seal which can be punctured by a needle to enable a needle to pass through this seal and through the inlet, and which is self-sealing upon removal of the needle, the valve member being operable between the open and closed position when the needle passes through the seal, the rear opening of the tubular member communicating with the outlet of the valve, and a needle which passes through the valve and through the tubular member.

13. A cannula/catheter assembly comprising an elongate tubular member adapted for insertion into a body cavity, the tubular member having a front opening and a rear opening, a valve comprising a valve body having an inlet to allow a fluid to pass into the valve body, an outlet to allow fluid to exit the valve, a valve member which is movable between a closed position where fluid is prevented from passing through the valve body, and an open position where fluid can pass through the valve body, the valve member including a seal which can be punctured by a needle to enable a needle to pass through this seal and through the inlet, and which is self-sealing upon removal of the needle, the valve member being operable between the open and closed position when the needle passes through the seal, the rear opening of the tubular member communicating with the outlet of the valve, a needle which passes through the valve and through the tubular member, and a needle retracting device attached to the needle, the retracting device able to retract the needle after the cannula has been inserted.
